(19) United States
(12) Patent Application Publication
(10) Pub. No.: US 2025/0050078 A1
(43) Pub. Date: Feb. 13, 2025

(54) Balloon Dilation Devices

(71) Applicant: Entellus Medical, Inc., Plymouth, MN (US)

(72) Inventors: Anthony J. Hanson, Chaska, MN (US); Ryan Cerepak, Champlin, MN (US); Aaron Swandal, Plymouth, MN (US); John Drontle, Buffalo, MN (US)

(21) Appl. No.: 18/723,060

(22) PCT Filed: Jun. 28, 2022
(86) PCT No.: PCT/US2022/035321

§ 371 Date: Jun. 21, 2024

Related U.S. Application Data
(60) Provisional application No. 63/294,058, filed on Dec. 27, 2021.
(60) Provisional application No. 63/332,290, filed on Apr. 19, 2022.

(51) Int. Cl. A61M 29/02

(52) U.S. Cl. CPC A61M 29/02

(57) ABSTRACT

A balloon dilation device includes a handle (110) that defines an internal cavity (116), a shaft (118) extending distally from the distal end of the handle, an inflatable balloon (126) disposed about the distal end of the shaft, and an inflation lumen (142) extending from an inflation port (144) of the handle to the inflatable balloon. The inflatable balloon is configured to be actuated between a contracted state and an expanded state. The shaft includes a vent lumen extending between a first opening at a distal end of the shaft and a second opening of the shaft is disposed in the internal cavity of the handle. A vent lumen of the shaft and a vent opening (158) in an exterior surface of the handle define an airflow pathway between the first opening and the vent opening.

### CROSS-REFERENCE TO RELATED APPLICATIONS

The present application claims the benefit of U.S. Provisional Patent Application No. 63/332,290, filed Apr. 19, 2022, and U.S. Provisional Patent Application No. 63/294,058, filed Dec. 27, 2021, the contents of which are hereby incorporated by reference in their entireties.

### FIELD

The present disclosure generally relates to balloon dilation devices and, in particular, a balloon dilation device for dilating and ventilating a Eustachian Tube.

### BACKGROUND

Eustachian tubes are relatively small anatomical passageways that extend between the middle ears and the nasopharynx. When properly functioning, the Eustachian tubes are primarily responsible for equalizing ear pressure and draining fluid from the middle ear. For example, the Eustachian tubes can open and close responsive to chewing, swallowing, or yawning to allow air flow between the middle of the ear and the throat and/or the nose.

Eustachian tube dysfunction (ETD) is a condition that occurs when one or both of the Eustachian tubes can become partially or completely blocked. ETD can cause pain, hearing difficulties, and/or discomfort. In some cases, ETD may resolve without treatment or with at-home treatments such as nasal sprays and/or antihistamine tablets. In other cases, ETD may persist or reoccur despite at-home treatments.

A treatment for ETD is a Eustachian tube balloon dilation procedure. During the procedure, a balloon dilation device is inserted through the nose, through the nasopharynx to the Eustachian tube opening, alternatively referenced to as an ostium pharyngeum, and into the Eustachian tube. While an inflatable balloon of the balloon dilation device is positioned in the Eustachian tube, the inflatable balloon is inflated to open the Eustachian tube. After the Eustachian tube is opened, the inflatable balloon is deflated and removed. As a result of this procedure, the Eustachian tube generally is able to open and air can again flow through Eustachian tube.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1A depicts a simplified block diagram of a balloon dilation device, according to an example.

FIG. 1B depicts a side view of the balloon dilation device shown in FIG. 1A, according to an example.

FIG. 1C depicts a top view of the balloon dilation device shown in FIG. 1A, according to an example.

FIG. 1D depicts a partially exploded view of the balloon dilation device shown in FIG. 1A, according to an example.

FIG. 1E depicts a first cross-sectional view of the balloon dilation device shown in FIG. 1C, according to an example.

FIG. 1F depicts a second cross-sectional view of the balloon dilation device shown in FIG. 1C, according to an example.

FIG. 1G depicts a third cross-sectional view of the balloon dilation device shown in FIG. 1C, according to an example.

FIG. 2 depicts a shaft of a balloon dilation device, according to an example.

FIG. 3A depicts a top view of a balloon dilation device with an inflatable balloon in a proximal-most position, according to an example.

FIG. 3B depicts a top view of a balloon dilation device of FIG. 3A with the inflatable balloon in a distal-most position, according to an example.

FIG. 4A depicts a side view of the balloon dilation device of FIG. 3A with an inflatable balloon in the proximal-most position, according to an example.

FIG. 4B depicts a side view of a balloon dilation device of FIG. 3A with the inflatable balloon in the distal-most position, according to an example.

FIG. 5A depicts a side view of the balloon dilation device of FIG. 3A with an inflatable balloon in the proximal-most position, according to an example.

FIG. 5B depicts a side view of a balloon dilation device of FIG. 3A with the inflatable balloon in the distal-most position, according to an example.

FIG. 6A depicts a side view of a first sleeve portion of a sleeve, according to an example.

FIG. 6B depicts an end view of a distal end of the first sleeve portion shown in FIG. 6A, according to an example.

FIG. 6C depicts an end view of a proximal end of first sleeve portion shown in FIG. 6A, according to an example.

FIG. 6D depicts an enlarged side view of the distal end and the proximal end of first sleeve portion shown in FIG. 6A, according to an example.

FIG. 7A depicts a side view of a second sleeve portion of a sleeve, according to an example.

FIG. 7B depicts an end view of the second sleeve portion shown in FIG. 7A, according to an example.

FIG. 8 depicts a subassembly of the first sleeve portion shown in FIGS. 6A-6D, the second sleeve portion shown in FIGS. 7A-7B, and an inflatable balloon, according to an example.

FIG. 9 depicts the subassembly of FIG. 8 after forming an atraumatic tip on a distal end of the inflatable balloon, according to an example.

FIG. 10 depicts a flowchart for a method of using a balloon dilation device, according to an example.

FIG. 11 depicts a flowchart for a method of using a balloon dilation device that can be used with at least the method depicted in FIG. 10, according to an example.

FIG. 12 depicts a flowchart for a method of using a balloon dilation device that can be used with at least the method depicted in FIG. 10, according to an example.

FIG. 13 depicts a flowchart for a method of using a balloon dilation device that can be used with at least the method depicted in FIG. 10, according to an example.

FIG. 14 depicts a flowchart for a method of using a balloon dilation device that can be used with at least the method depicted in FIG. 10, according to an example.

FIG. 15 depicts a flowchart for a method of using a balloon dilation device that can be used with at least the method depicted in FIG. 10, according to an example.

FIG. 16 depicts a flowchart for a method of using a balloon dilation device that can be used with at least the method depicted in FIG. 10, according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, an inflatable balloon of a balloon dilation device can be inflated to dilate a Eustachian tube and treat ETD. When the inflatable balloon is inflated, the inflatable balloon may contact the Eustachian tube around a substantial portion or an entirety of a circumference of the inflatable balloon. In these instances, there may little or no space between an exterior of the inflatable balloon and the Eustachian tube for air to flow through the Eustachian tube. If airflow was blocked while the inflatable balloon is inflated in the Eustachian tube, pressure may build up at the middle ear and this may present a risk of potential damage to the tympanic membrane in the middle ear.

To mitigate this risk, the balloon dilation device is configured to provide an airflow pathway around or through the inflatable balloon while the inflatable balloon is inflated and in contact with the Eustachian tube. One approach involves the balloon dilation device including a tube that extends through the inflatable balloon, and vent openings in sidewalls of the tube on opposing sides of the inflatable balloon. In this arrangement, air can enter the vent opening immediately distal of the inflation balloon and exit the vent opening immediately proximal of the inflation balloon. However, this approach suffers from several drawbacks. For example, because the vent openings are adjacent to the inflatable balloon, the vent openings are positioned in the anatomy of the patient (e.g., in Eustachian tube). As a result, the vent openings may be subject to clogging due to, for instance, fluids and/or tissue contacting the vent openings. This risk may be further exacerbated due to one or more of the vent openings being in the sidewall of the tube, which faces the wall of the Eustachian tube.

Another approach to providing an airflow pathway involves the balloon dilation device including a vent lumen that extends from a distalmost end of the balloon dilation device to a port on a handle of the balloon dilation device. For example, one balloon dilation device includes the inflatable balloon mounted on a guide tube, which extends from a position distal of the inflatable balloon to a port on a proximal portion of the handle. The guide tube defines a lumen that can be used as an airflow passageway from a portion of the Eustachian tube that is distal of the inflatable balloon to the port, which is located outside of the patient's anatomy. As such, this approach can beneficially mitigate the problems associated with clogged vent openings in the sidewall of the tube.

However, specifically in the context of a Eustachian tube balloon dilation procedure, the approach of the balloon dilation device providing the airflow passageway along the guide tube to the port accessible on a proximal portion of the handle can be improved upon. For example, in the arrangement of the port and the guide tube described above, the port and the lumen of the guide tube provide direct access to the anatomy at the distal end of the guide tube. When treating a sinus outflow tract, this may beneficially facilitate aspirating the anatomy at the distal end of the guide tube, delivering a fluid to the distal end of the guide tube, and/or positioning a device that can assist in determining the location of the distal end of the guide tube (e.g., a guide wire, an image guided surgery sensor, and/or an optical fiber). While these operations may be beneficial for treating a sinus outflow tract, performing these operations using the lumen of the guide tube while treating the Eustachian tube may block the airflow passageway and cause undesirable pressure build up at the middle ear. As such, it would be beneficial for the balloon dilation device to provide the airflow passageway from a location in the Eustachian tube that is distal of the inflatable balloon to a location outside of the patient's anatomy (e.g., the Eustachian tube and a nasal cavity) while inhibiting or preventing the lumen of the guide tube from being used for operations other than ventilating the Eustachian tube.

The present application provides for a balloon dilation device that can address one or more of the challenges described above. In an example, the balloon dilation device includes a handle, a shaft extending distally from a distal end of the handle, and an inflatable balloon disposed about a distal end of the shaft. The shaft can include a vent lumen extending between a first opening at the distal end of the shaft and a second opening of the shaft, which is disposed in the internal cavity of the handle. In this arrangement, at least the vent lumen of the shaft and a vent opening in an exterior surface of the handle define an airflow pathway between the first opening and the vent opening for ventilating the Eustachian tube while the inflatable balloon is in an expanded state.

Within examples, the vent lumen and the vent opening are configured to inhibit or prevent direct access to the vent lumen from the vent opening. For instance, to inhibit or prevent access to the vent lumen from the vent opening, the vent lumen and the vent opening can be configured such that the airflow pathway defines a tortuous path that inhibits access to the vent lumen. Additionally or alternatively, to inhibit or prevent access to the vent lumen from the vent opening, a longitudinal axis of the vent lumen can be transverse to the airflow pathway at the vent opening in the exterior surface of the handle. Additionally or alternatively, to inhibit or prevent access to the vent lumen from the vent opening, the second opening of the vent lumen can be proximal of the vent opening in the exterior surface of the handle.

In this arrangement, the balloon dilation device provides the airflow pathway from a location in the Eustachian tube that is distal of the inflatable balloon to a location of the handle that is external to the patient's anatomy (e.g., the Eustachian tube and a nasal cavity), while also inhibiting or preventing an operator from using the vent tube for a purpose other than ventilating the Eustachian tube.

Additionally, in the context of Eustachian tube dilation procedures, another challenge with existing balloon dilation devices relates to the process of navigating the balloon dilation device to the Eustachian tube. As noted above, some existing balloon dilation devices are designed to treat a variety of anatomies including, for example, one or more sinus outflow tracts (e.g., of the frontal sinus, the maxillary sinus, and/or the sphenoid sinus) as well as the Eustachian tube. In some instances, due to variances in patient anatomy, a shape of a distal portion of the balloon dilation device may present challenges in navigating around the anatomy to access the Eustachian tube. As described in further detail below, the present application provides for a balloon dilation device that can additionally or alternatively address one or more of the challenges associated with navigating the balloon dilation device to the Eustachian tube.

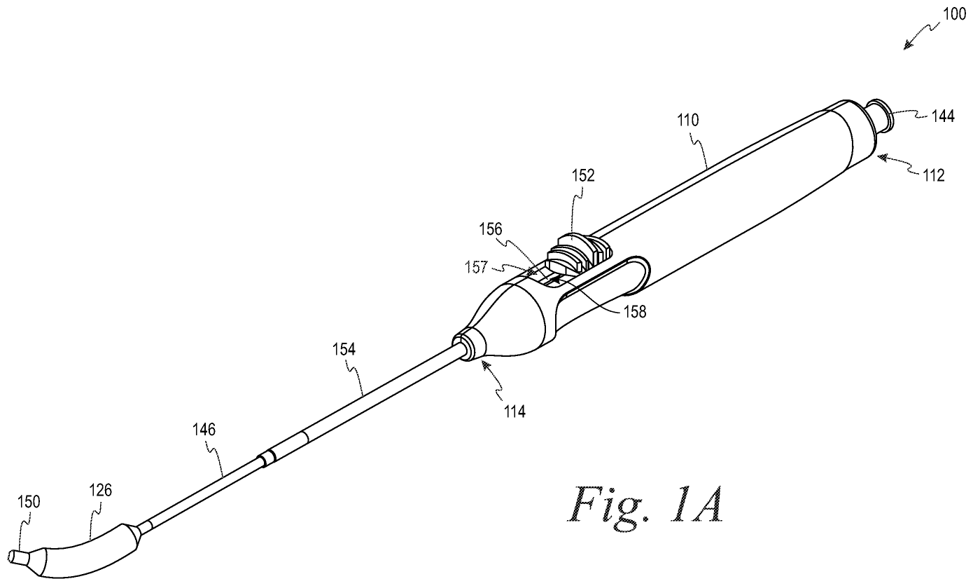

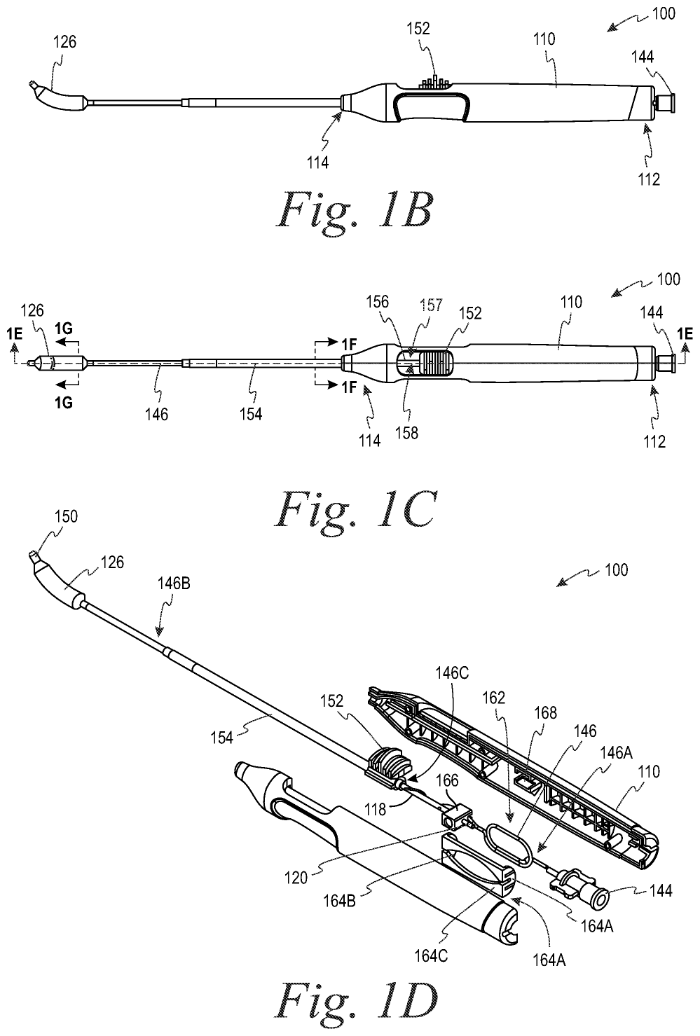

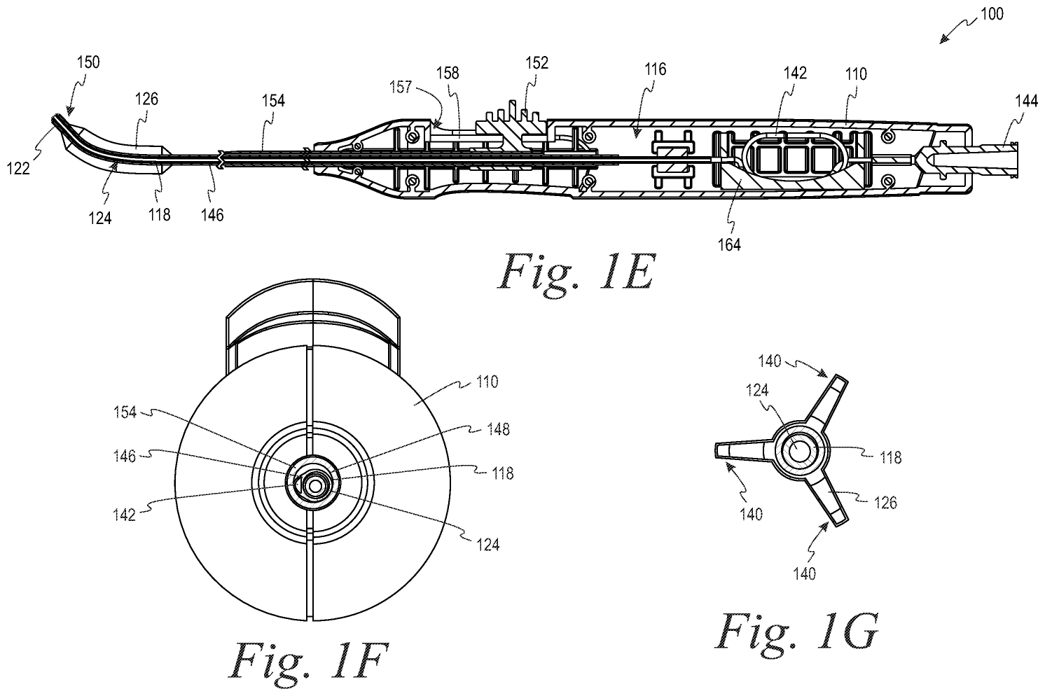

Referring now to FIGS. 1A-1G, a balloon dilation device 100 is shown according to an example. In particular, FIG. 1A depicts a perspective view of the balloon dilation device 100, FIG. 1B depicts a side view of the balloon dilation device 100, FIG. 1C depicts a top view of the balloon dilation device 100, FIG. 1D depicts a partially exploded view of the balloon dilation device 100, FIG. 1E depicts a cross-sectional view of the balloon dilation device 100 through line 1E in FIG. 1C, FIG. 1F depicts a cross-sectional view of the balloon dilation device through line 1F in FIG. 1C, and FIG. 1G depicts a cross-sectional view of the balloon dilation device through line 1G in FIG. 1C, according to an example.

As shown in FIGS. 1A-1F, the balloon dilation device 100 can include a handle 110 having a proximal end 112 and a distal end 114. In general, the handle 110 can be configured to facilitate a user gripping and manipulating the balloon dilation device 100 while performing a Eustachian tube dilation procedure. For example, the handle 110 can have a shape and/or a size that can facilitate a user performing the procedure by manipulating the balloon dilation device 100 using a single hand. In one implementation, the handle 110 can have a shape and/or a size that facilitates the user holding the balloon dilation device 100 using a writing utensil grip.

As shown in FIG. 1D, the handle 110 can define an internal cavity 116. As described in further detail below, the internal cavity 116 can house one or more components of the balloon dilation device 100. In FIG. 1D, the handle 110 is formed from a plurality of segments that are fixedly coupled together (e.g., by a welding coupling, an adhesive coupling, and/or a fastener coupling). This can help to assemble components within the internal cavity 116 during a manufacturing process. However, in another example, the handle 110 can be formed as a single, monolithic structure that defines the internal cavity 116.

As shown in FIGS. 1D-1F, the balloon dilation device 100 also includes a shaft 118 extending distally from the distal end 114 of the handle 110. More specifically, the shaft 118 extends between a proximal end 120 (shown in FIG. 1D) located in the internal cavity 116 of the handle 110 and a distal end 122 (shown in FIG. 1E) located external to the handle 110. The shaft 118 can be fixedly coupled to the handle 110 such that the shaft 118 and the handle 110 cannot move relative to each other. The shaft 118 may include a strengthening sleeve disposed over a smaller diameter core portion and fixed thereto for all or a portion of a length of the shaft 118 to provide the shaft 118 with an increased resistance to bending and buckling.

The shaft 118 includes a vent lumen 124 (shown in FIG. 1E) extending between a first opening at the distal end 122 of the shaft 118 and a second opening of the shaft 118, which is disposed in the internal cavity 116 of the handle 110. For example, as shown in FIG. 1D, the second opening of the shaft 118 can be at the proximal end 120 of the shaft 118. However, in another example, the second opening of the shaft 118 can include one or more openings located at the proximal end 120 of the shaft 118 and/or in a sidewall of the shaft 118. The second opening of the shaft 118 and a cross sectional area of the vent lumen 124 may be of a size substantially the same as the first opening at the distal end 122, or greater, to allow any debris that has entered the vent lumen 124 at the distal end 122 to pass freely along the vent lumen 124 and through the second opening. As described in further detail below, the vent lumen 124 of the shaft 118 can define at least a portion of an airflow passageway for ventilating the Eustachian tube.

As shown in FIG. 1E and FIG. 1G, the balloon dilation device 100 can further include an inflatable balloon 126 disposed about the distal end 122 of the shaft 118. In this arrangement, the shaft 118 can help to provide underlying support for at least a portion of the inflatable balloon 126. For example, the shaft 118 can be formed from a substantially rigid material such as, for instance, a stainless steel hypotube. Forming the shaft 118 from a substantially rigid material can additionally or alternatively assist in providing tactile feedback to a user holding the handle 110 while inserting the inflatable balloon 126 through a nasal cavity to the Eustachian tube.

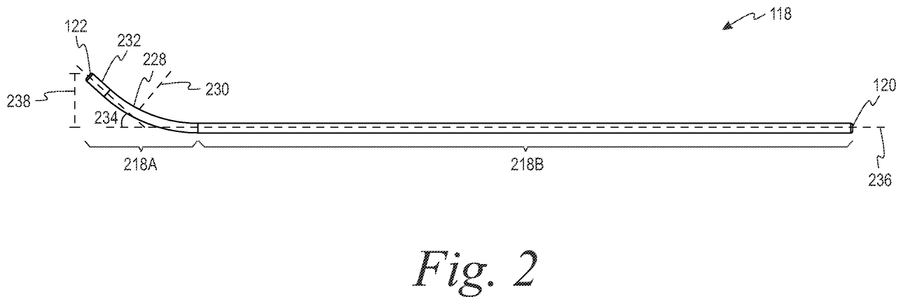

In some examples, the shaft 118 can have a shape and/or a size that assists in navigating the distal end 122 of the shaft 118 and the inflatable balloon 126 around anatomical structures of the patient while transnasally inserting the balloon dilation device 100 to the Eustachian tube. FIG. 2 depicts a side view of the shaft 118 according to an example. For instance, as shown in FIG. 2, a distal portion 218A of the shaft 118 can include a curved portion 228 that can help to navigate the distal end 122 of the shaft 118 and the inflatable balloon 126 through the nasal cavity to the Eustachian tube.

In one example, the curved portion 228 can have a radius of curvature 230 within a range of approximately 0.5 inches to approximately 1.0 inches (i.e., approximately 12.7 mm to approximately 25.4 mm) such as, for example, approximately 0.7 inches (i.e., approximately 17.78 mm). Additionally or alternatively, the distal portion 218A of the shaft 118 can include a straight segment 232 extending from the curved portion 228 to the distal end 122. An angle 234 between a tangent line tangent to the distal portion 218A at the distal end 122 and a longitudinal axis 236 of a proximal portion 218B of the shaft 118 can be within an example range of approximately 30 degrees to approximately 60 degrees, with one example angle being approximately 45 degrees. When the distal portion 218A includes the straight segment 232, the tangent line is parallel to the straight segment. Additionally or alternatively, the distal portion 218A can be configured such that the distal end 122 of the shaft 118 is radially offset 238 from the longitudinal axis 236 within an example range of approximately 6 millimeters (mm) to approximately 10 mm, with one example being approximately 8 mm. Additionally or alternatively, the straight segment 232 can have an example length in an example range of approximately 2 mm to 6 mm, with one example length being approximately 4 mm. It has been found that an implementation in which the distal portion 218A of the shaft 118 includes such a combination of the offset 238 and the angle 234 can improve navigating the inflatable balloon 126 to the Eustachian tube by reducing or mitigating clearance issues when navigating around common nasal anatomical structures, including, by way of example, the inferior turbinate, and a nasal irregularity including by way of example, a septal deviation and a septal spur, in the nasal cavity of patients. As such, this example configuration of the distal portion 218A of the shaft 118 can provide improvements over some existing balloon dilation devices that are designed to treat a variety of anatomies including, for example, one or more sinus outflow tracts (e.g., of the frontal sinus, the maxillary sinus, and/or the sphenoid sinus) as well as the Eustachian tube.

In some implementations, the shaft 118 can be non-malleable at the distal portion 218A, including at the curved portion 228. In such implementations, the shape of the shaft 118 is fixed and cannot be changed without compromising a structural integrity of the shaft 118 (e.g., without forming a kink in the shaft 118). This can assist in inhibiting or preventing a user from altering the shaft 118 to have a shape that is not well suited for accessing the Eustachian tube and/or to inhibit a user from using the balloon dilation device 100 for treating an anatomical structure other than the Eustachian tube. Additionally, making the shaft 118 non-malleable at the distal portion 218A can help to discourage an operator from attempting to reshape the distal portion 218A in a manner that may compromise the integrity of the inflatable balloon 126.

In other implementations, the shaft 118 can be malleable at the distal portion 218A. This may allow a user to modify the shape of the distal portion 218A according to a specific anatomy of a particular patient and/or adapt the balloon dilation device 100 for treating an anatomical structure other than the Eustachian tube. In an example of an implementation in which the shaft 118 is malleable at the distal portion 218A, a manufacturing process can include a step of annealing the distal portion 218A of the shaft 118 to impart malleability.

In some examples in which the distal portion 218A of the shaft 118 is malleable or non-malleable, the curved portion 228 can be formed during the manufacturing process and prior to a user preparing to use the balloon dilation device 100. In such examples, the balloon dilation device 100 can be preset to perform the Eustachian tube dilation procedure without any modifications to the shaft 118 by the user. In other examples in which the distal portion 218A of the shaft 118 is malleable, the shaft 118 can be manufactured without the curved portion 228, and the user can form the curved portion 228 by bending the distal portion of the shaft 118 prior to inserting the balloon dilation device 100 into the nasal cavity of the patient. However, as described above, implementations in which the shaft 118 is non-malleable may provide benefits relative to implementations in which the shaft 118 is malleable (e.g., mitigating a risk of damage to the inflatable balloon 126 resulting from a bending process).

As described above, the inflatable balloon 126 is disposed about the distal end 122 of the shaft 118. The inflatable balloon 126 is configured to be actuated between a contracted state and an expanded state. FIGS. 1A, 1C, and 1G show the inflatable balloon 126 in the contracted state, according to an example. As shown in FIG. 1G, the inflatable balloon 126 can be folded into a plurality of flaps 140 during the manufacturing process. The flaps 140 and folds can help to insert the inflatable balloon 126 into a balloon protector tube that can cover and protect the inflatable balloon 126 until the balloon dilation device 100 is ready to be used on the patient.

FIGS. 1B, 1D, and 1E show the inflatable balloon 126 in the expanded state. In this example, the inflatable balloon 126 has a cylindrical shape when the inflatable balloon 126 is inflated to the expanded shape. For example, a cross-sectional shape of the inflatable balloon 126 through line 1G can be a circle when the inflatable balloon 126 is in the expanded state. This shape of the inflatable balloon 126 can help to increase (or maximize) an amount of contact between the inflatable balloon 126 and the Eustachian tube when the inflatable balloon 126 is inflated to the expanded state.

In one example, the inflatable balloon 126 can have a cross-sectional diameter within the range of about 3 mm to about 9 mm when inflated to the expanded state. In another example, the inflatable balloon 126 can have a diameter within the range of about 5 to about 7 mm when inflated to the expanded state. In an example, the inflatable balloon 126 can have a length within the range of about 10 mm to 25 mm. These dimensions may be beneficial for treating the Eustachian tube of most patients.

As shown in FIGS. 1B, 1D, and 1E, the inflatable balloon 126 can be formed from a material having a flexibility that allows the inflatable balloon 126 to extend along and around the curved portion 228 of the shaft 118. In some examples, the inflatable balloon 126 can be formed of one or more high strength and flexible polymeric materials such as, for instance, one or more polyamides (e.g., Nylon) and/or one or more elastomers (e.g., PEBAX). In one implementation of the manufacturing process, the inflatable balloon 126 can be “blow molded” to a relatively thin wall thickness, and capable of holding relatively high pressures from about 6 atmospheres to about 20 atmospheres of inflation pressure.

To actuate the inflatable balloon 126 between the contracted state and the expanded state, the balloon dilation device 100 includes an inflation lumen 142 extending from an inflation port 144 of the handle 110 to the inflatable balloon 126. The inflation lumen 142 defines a fluid channel between the inflation port 144 on the handle 110 and an interior of the inflatable balloon 126. The inflation port 144 can include a connection interface for fluidically coupling with an inflation device such as, for instance, a syringe that can supply a fluid (e.g., saline or water) to the inflation port 144. As an example, the inflation port 144 can include a Luer connection interface for coupling the balloon dilation device 100 to the inflation device. In this arrangement, when the inflation device is coupled to the inflation port 144, the inflation device can be operated to (i) supply the fluid along the inflation lumen 142 to the inflatable balloon 126 and inflate the inflatable balloon 126 to the expanded state, and (ii) remove, along the inflation lumen 142, the fluid from the inflatable balloon 126 to deflate the inflatable balloon 126 to the contracted state.

In the example shown in FIGS. 1D-1F, the inflation lumen 142 is external to the vent lumen 124 of the shaft 118. This can help to provide better airflow through the vent lumen 124 of the shaft 118 as opposed to an alternative example in which the inflation lumen 142 is disposed in the shaft 118. In the illustrated example, the inflation lumen 142 can be defined by a sleeve 146 having a first end that is fluidically coupled to the inflation port 144 and a second end that is fluidically coupled to the inflatable balloon 126.

In an example, as shown in FIGS. 1D-1F, the sleeve 146 can include a plurality of sections having different configurations. For instance, the sleeve 146 can include a proximal section 146A that defines a single lumen, which provides the inflation lumen 142. The sleeve 146 can also include a distal section 146B having two lumens. As shown in FIG. 1F, one lumen of the distal section 146B can provide the inflation lumen 142 and the other lumen of the distal section 146B can be a rider lumen 148 that receives the shaft 118. As such, in the illustrated example, the distal section 146B of the sleeve 146 can be disposed around the shaft 118. Also, as shown in FIG. 1F, at least a portion of the inflation lumen 142 and the vent lumen 124 can be in a side-by-side arrangement (e.g., extending along parallel longitudinal axes).

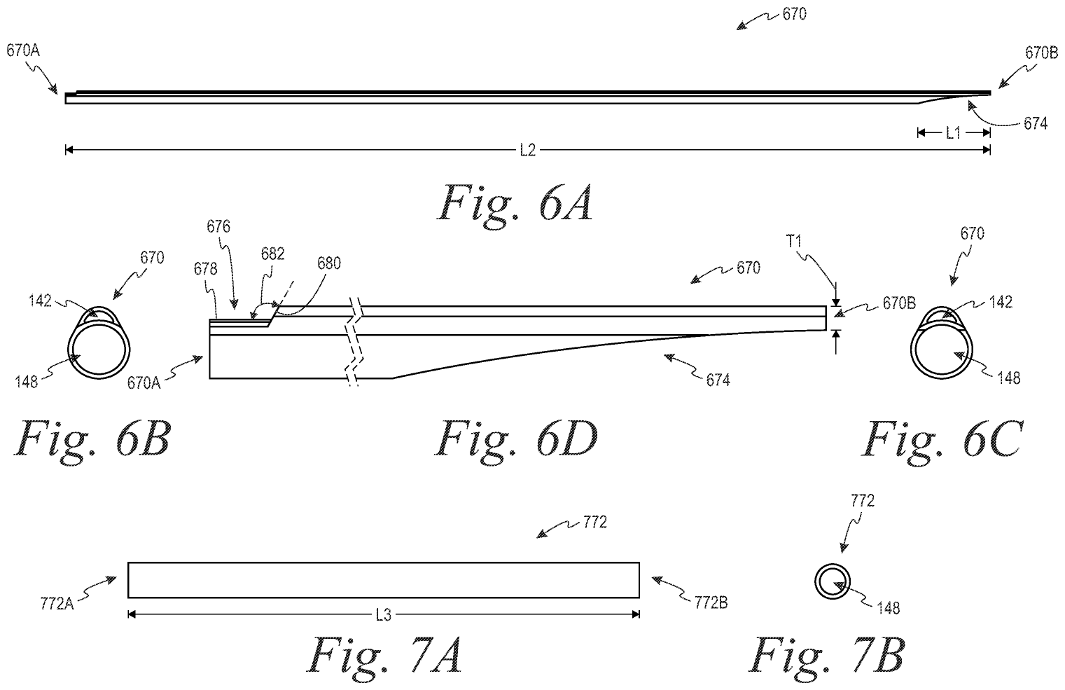

As shown in FIG. 1D, the sleeve 146 can also include a transition section 146C for transitioning between the different configurations of the proximal section 146A and the distal section 146B of the sleeve 146. The transition section 146C can be formed by “skiving” away a portion of the sleeve 146 to form the rider lumen 148 and extend around the shaft 118. Additional details relating to the inflatable balloon 126, the sleeve 146, and processes for forming the inflatable balloon 126 and the sleeve 146 are described in further detail below with respect to FIGS. 6A-9.

In an example, the rider lumen 148 can have a diameter that is slightly larger than an outer diameter of the shaft 118. As described in further detail below, the rider lumen 148 can thus enable the sleeve 146 to advance and retract over the shaft 118 in a close-fit arrangement. As examples, the outer diameter of the sleeve 146 can be in a range of about 0.050 inch to about 0.110 inch or in the range of about 0.070 inch to about 0.100 inch. In some examples, one or both of an exterior surface of the shaft 118 and an interior surface of the rider lumen 148 can be coated with a lubricious coating to reduce frictional contact forces.

As shown in FIGS. 1A-1E, the balloon dilation device 100 can additionally include an atraumatic tip 150 at a distalmost end of the inflatable balloon 126. The atraumatic tip 150 can help to provide for smooth movement of the balloon dilation device 100 through tight passages in the nasal cavity, and/or assist in providing tactile feedback to the user while navigating the inflatable balloon 126 to the Eustachian tube. As an example, the atraumatic tip 150 can have a bulbous shape and/or the atraumatic tip 150 can be formed of a relatively pliable material.

In FIGS. 1A-1G, the atraumatic tip 150 and the inflatable balloon 126 are integrally formed as a single, monolithic structure. For instance, the atraumatic tip 150 can be formed from excess material of the inflatable balloon 126 before, during, or after forming the inflatable balloon 126. In an example, the atraumatic tip 150 can have an outer diameter in a range between about 1 mm and about 3 mm. Also, as shown in FIGS. 1A-1G, the atraumatic tip 150 extends distally of the distal end 122 of the shaft 118. In this arrangement, the atraumatic tip 150 can provide a cushion between the distal end 122 of the shaft 118, which can be made of a substantially rigid material, and the anatomy of the patient.

FIG. 1G depicts the inflatable balloon 126 in the contracted state. As shown in FIG. 1G, the inflatable balloon 126 can be folded into a plurality of pleats. To help protect the inflatable balloon 126 between a time of manufacture and a time of use, the inflatable balloon 126 can be inserted in a protective sleeve while the inflatable balloon 126 is in the contracted state. Folding the inflatable balloon 126 into the pleats can help to safely insert the inflatable balloon into the protective sleeve. Additionally, upon deflation of an inflated balloon 126, the pleats facilitate a return of a shape of the deflated balloon 126 to a shape substantially resembling a pre-inflation shape of the balloon 126. The pleats thereby beneficially provide the deflated balloon 126 with a desired reduced crossing profile, that is, a reduced maximum diameter, facilitating both withdrawal of the balloon 126 and reinsertion of the balloon 126. When the inflatable balloon 126 is inflated to the expanded state, the pleats expand outwardly and unfold the pleats. For instance, as described above, the inflatable balloon 126 can have a cylindrical shape when the inflatable balloon 126 is inflated to the expanded shape as shown, for example, in FIGS. 1B, 1D, and 1E.

Although it can be beneficial to form the atraumatic tip 150 and the inflatable balloon 126 as a single, monolithic structure, the atraumatic tip 150 and the inflatable balloon 126 can be distinct structures that are coupled together in other examples.

In some examples, the inflatable balloon 126 can be fixedly coupled to the shaft 118 such that the inflatable balloon 126 cannot move relative to the shaft 118. This may simplify a manufacturing process and reduce costs. However, fixedly coupling the inflatable balloon 126 to the shaft 118 can make navigating the inflatable balloon 126 into the Eustachian tube more challenging. In other examples such at the example shown in FIGS. 1A-1E, the inflatable balloon 126 can be movable relative to the shaft 118. As described in further detail below, this can help with inserting the inflatable balloon 126 into the Eustachian tube by allowing for (i) initially navigating the distal end 122 of the shaft 118 and the inflatable balloon 126 to the Eustachian tube opening in the nasopharynx using the handle 110 and (ii) then moving the inflatable balloon 126 relative to the shaft 118 to insert the inflatable balloon 126 through the Eustachian tube opening and to a position in the Eustachian tube at which the inflatable balloon 126 can be actuated to the expanded state to dilate the Eustachian tube.

As shown in FIGS. 1A-1E, the balloon dilation device 100 can include a balloon advancer member 152 that is configured to move the inflatable balloon 126 relative to the shaft 118. For example, the balloon advancer member 152 can be coupled to the sleeve 146, and the sleeve 146 is coupled to the inflatable balloon 126. In this arrangement, moving the balloon advancer member 152 in a distal direction relative to the handle 110 moves the sleeve 146 and the inflatable balloon 126 distally relative to the shaft 118. Similarly, moving the balloon advancer member 152 in a proximal direction relative to the handle 110 moves the sleeve 146 and the inflatable balloon 126 proximally relative to the shaft 118.

In FIG. 1E, the balloon advancer member 152 is coupled to the sleeve 146 by a support tube 154, with the tube 154 communicating movement of the advancer member 152 to the sleeve 146. The support tube 154 and the sleeve 146 may move as a unit with movement of the balloon advancer member 152. The support tube 154 can be located about the external periphery of a portion of the sleeve 146 to impart further stiffness to the balloon dilation device 100. For example, the support tube 154 can be formed for a metallic material such as, for instance, a stainless steel hypotube.

In FIGS. 1A-1D, the support tube 154 can extend along a proximal portion of the sleeve 146, but not a distal portion of the sleeve 146. This can help to achieve a desired flexibility of the distal portion 218A of the shaft 118 and/or the distal portion of the sleeve 146, which may help to navigate the balloon dilation device 100 through the patient's anatomy during insertion. In other examples, the support tube 154 can extend over an entirety of the sleeve 146 between the handle 110 and the inflatable balloon 126. As shown in FIG. 1E, the support tube 154 may not extend over at least a portion of the sleeve 146 that is proximal of the balloon advancer member 152. This can help to allow the sleeve 146 to accommodate movement of the inflatable balloon 126 relative to shaft 118. However, in other examples, the balloon dilation device 100 can omit the support tube 154 and/or the balloon advancer member 152 can be directly coupled to the sleeve 146.

The balloon advancer member 152 can be configured enable a finger of the user (e.g., index finger or thumb) to easily move the balloon advancer member 152 relative to the handle 110. For example, as shown in FIGS. 1A and 1E, the balloon advancer member 152 can include a plurality of protrusions that form a generally convex surface for engaging a finger of the user. The discontinuity between the protrusions can help to provide tactile feel and/or mitigate slippage of the finger on the balloon advancer member 152. However, the balloon advancer member 152 can have a different shape and/or a different size in other examples. Additionally, although the balloon advancer member 152 is moveable along a top surface of the handle 110 (e.g., a surface facing a direction in which the curved portion 228 is bent) in the illustrated example, the balloon advancer member 152 can be disposed and moveable along a different surface of the handle 110 in other examples.

As shown in FIGS. 1A-1C and 1E, the balloon advancer member 152 can be configured to move within a slot 156 in the exterior surface of the handle 110. The slot 156 can extend in a direction that is parallel to the longitudinal axis 236 of the shaft 118. In some examples, a length of the slot 156 can define a range of motion of the inflatable balloon 126 relative to the shaft 118. For instance, a distal end of the slot 156 can provide a distal stop to define a distalmost position of the inflatable balloon 126 relative to the shaft 118 and/or a proximal end of the slot 156 can define a proximal stop to define a proximal-most position of the inflatable balloon 126 relative to the shaft 118. In other examples, such as the example shown in FIG. 1E, the slot 156 and the balloon advancer member 152 can be positioned in a recess 157 of the handle 110, and the range of motion of the inflatable balloon 126 relative to the shaft 118 can be defined by a length of the recess 157 and a length of the balloon advancer member 152. For instance, a distal edge of the balloon advancer member 152 can engage a distal edge of the recess 157 when the inflatable balloon 126 is in the distalmost position, and a proximal edge of the balloon advancer member 152 can engage a proximal edge of the recess 157 when the inflatable balloon 126 is in the proximal-most position. In still other examples, the range of motion of the inflatable balloon 126 can be defined by a combination of the length of the slot 156, the length of the recess 157, and/or the length of the balloon advancer member 152.

In one example, the range of motion can be approximately 10 millimeters. This range of motion can allow a user to comfortably move the balloon advancer member 152 between the distalmost position and the proximal-most position of the inflatable balloon 126 without having to adjust a grip on the handle 110. In another example, the range of motion can be a range between approximately 0.25 inch and approximately 1.50 inches.

As noted above, the balloon dilation device 100 provides an airflow pathway from a location in the Eustachian tube that is distal of the inflatable balloon 126 to a location of the handle 110 that is external to the patient's anatomy (e.g., the Eustachian tube and a nasal cavity), while also inhibiting or preventing an operator from using the vent lumen 124 for a purpose other than ventilating the Eustachian tube. Additionally, as described above, the vent lumen 124 of the shaft 118 can define at least a portion of the airflow passageway for ventilating the Eustachian tube.

As shown in FIG. 1A, FIG. 1C, and FIG. 1E, the balloon dilation device 100 can also include a vent opening 158 in an exterior surface of the handle 110 to further define the airflow pathway. In some examples, the vent opening 158 can be disposed between the proximal end 112 of the handle 110 and the distal end 114 of the handle 110. Additionally or alternatively, the vent opening 158 can be nearer to the distal end 114 of the handle 110 than the proximal end 112 of the handle 110. As described in further detail below, this can help to inhibit access to the second opening of the shaft 118 and the vent lumen 124, which are disposed in the internal cavity 116 of the handle 110. More generally, inhibiting access to the second opening of the shaft 118 in the internal cavity 116 can (i) mitigate a risk of inadvertently blocking the vent lumen 124, and (ii) allow air to flow along the vent lumen 124 from the Eustachian tube into at least the internal cavity 116 regardless of a manner in which the handle 110 is grasped by an operator.

In the illustrated example, the slot 156 in the exterior surface of the handle 110 defines the vent opening 158 in the exterior surface of the handle 110. This can reduce (or minimize) the number of openings in the exterior surface of the handle 110 as compared to other examples in which the vent opening 158 includes one or more other openings providing only the venting functionality of the vent opening 158. However, in other examples, the vent opening 158 can include one or more other openings in the exterior surface of the handle 110 (e.g., in an implementation in which the balloon dilation device 100 omits the balloon advancer member 152 and/or the slot 156).

The vent lumen 124 and the vent opening 158 are configured to inhibit or prevent direct access to the vent lumen 124 from the vent opening 158. For example, as shown in FIG. 1E, to inhibit or prevent access to the vent lumen 124 from the vent opening 158, the vent lumen 124 and the vent opening 158 can be configured to have at least one of feature selected from a group of features consisting of: (i) the airflow pathway defines a tortuous path that inhibits access to the vent lumen 124, (ii) a longitudinal axis of the vent lumen 124 can be transverse to the airflow pathway at the vent opening 158 in the exterior surface of the handle 110, and (ii) the second opening of the vent lumen 124 can be proximal of the vent opening 158 in the exterior surface of the handle 110.

Referring to FIG. 1E, for instance, the airflow pathway can be defined by the atraumatic tip 150, the inflatable balloon 126, the vent lumen 124, the internal cavity 116 of the handle 110, and the vent opening 158. In this arrangement, while the inflatable balloon is in the expanded state, air can flow through an inner lumen defined by the atraumatic tip 150 and the inflatable balloon 126 into the vent lumen 124 of the shaft 118. The air can then flow along the vent lumen 124 and out the second opening of the shaft 118 into the internal cavity 116 of the handle 110. To this point, the airflow pathway between the atraumatic tip 150 and the second opening of the shaft 118 can be substantially linear (e.g., along a proximal direction that is parallel to the longitudinal axis 236 of the shaft 118). However, when the air exits the second opening of the shaft 118 into the internal cavity 116, the airflow pathway turns back in a distal direction (e.g., turning approximately 180 degrees) towards the vent opening 158. The airflow pathway then turns again in a direction that is transverse to the longitudinal axis 236 and out the vent opening 158. This tortuous path along a relatively small space in the internal cavity 116 of the handle 110 can help to inhibit (or prevent), for example, a guide wire from being inserted into the vent lumen 124 from the vent opening 158.

Accordingly, the arrangement of the vent lumen 124 and the vent opening 158 of the balloon dilation device 100 can improve patient safety and reduce a risk of operator error in the context of Eustachian tube dilation procedures by inhibiting or preventing access to the vent lumen 124 from outside the handle 110 of the balloon dilation device 100. Additionally, in examples in which the balloon dilation device 100 includes only a single port (e.g., the inflation port 144), operation of the balloon dilation device 100 can be simplified in that the user does not need to determine which of multiple ports on the handle 110 should be used for inflating the inflatable balloon 126.

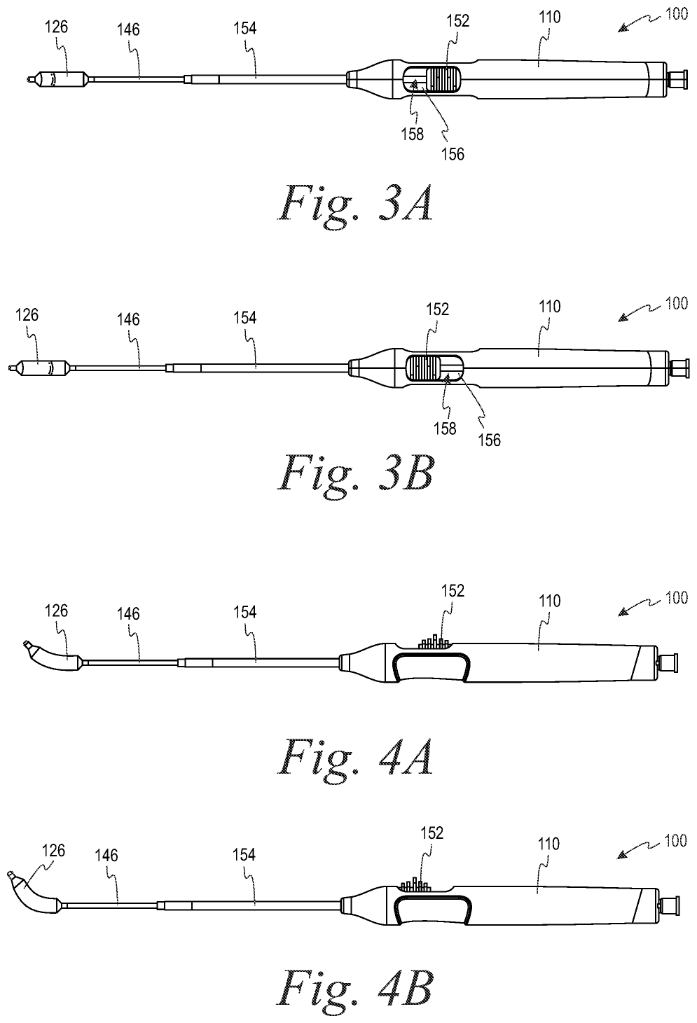

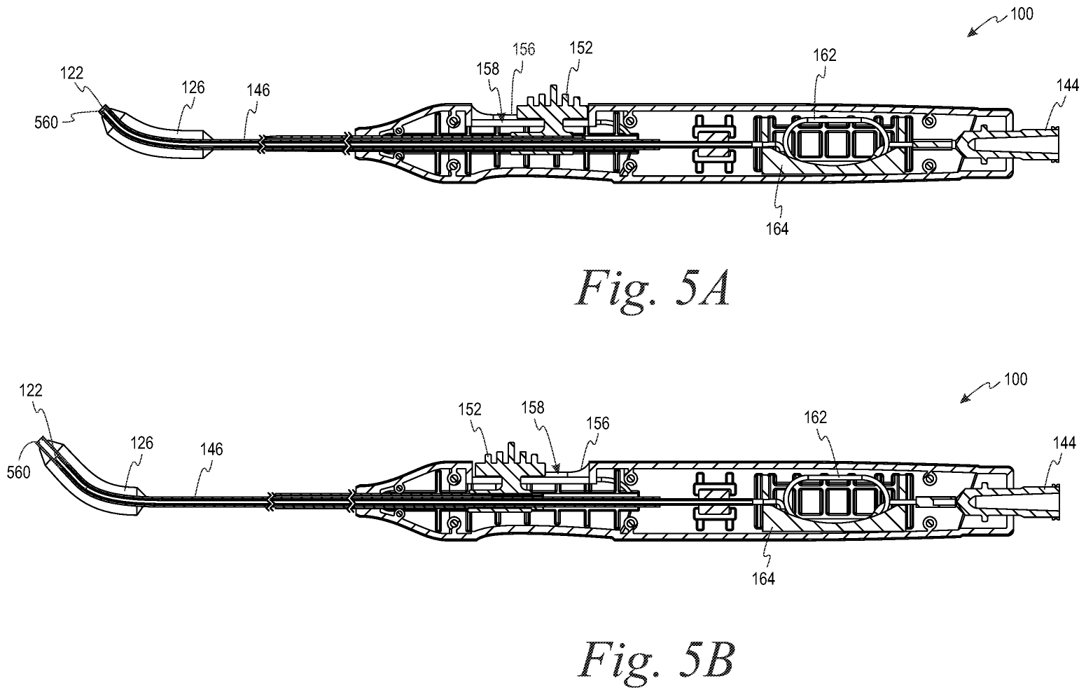

As described above, the inflatable balloon 126 can be moveable between a distal-most position and a proximal-most position relative to the shaft 118 and/or the handle 110. FIGS. 3A-5B show the balloon dilation device 100 with the balloon advancer member 152, the sleeve 146, and the inflatable balloon 126 in a plurality of different positions relative to the handle 110 and/or the shaft 118. More particularly, FIG. 3A, FIG. 4A, and FIG. 5A depict the balloon dilation device 100 with the inflatable balloon 126 in the proximal-most position relative to the shaft 118 and/or the handle 110. FIG. 3B, FIG. 4B, and FIG. 5B depict the balloon dilation device 100 with the inflatable balloon 126 in the distal-most position relative to the shaft 118 and/or the handle 110.

As shown in FIGS. 3A, 4A, and 5A, when the balloon advancer member 152 is in a proximal-most position in the slot 156 and the inflatable balloon 126 is in the proximal-most position relative to the shaft 118, the vent opening 158 is provided by a distal portion of the slot 156 that is uncovered by the balloon advancer member 152. As shown in FIGS. 3B, 4B, and 5B, when the balloon advancer member 152 is in a distal-most position in the slot 156 and the inflatable balloon 126 is in the distal-most position relative to the shaft 118, the vent opening 158 is provided by a proximal portion of the slot 156 that is uncovered by the balloon advancer member 152. Accordingly, in the illustrated implementation in which the slot 156 defines the vent opening 158, the vent opening 158 remains open for ventilating the Eustachian tube in all positions of the balloon advancer member 152 in the slot 156. As such, the balloon dilation device 100 can provide for ventilating the Eustachian tube in all positions of the inflatable balloon 126 relative to the shaft 118 and/or the handle 110.

As shown in FIGS. 5A-5B, the inflatable balloon 126 can include an internal shoulder 560 that faces the distal end 122 of the shaft 118. As shown in FIG. 5A, the internal shoulder 560 can abut against the distal end 122 of the shaft 118 when the inflatable balloon 126 is in the proximal-most position in some examples. This can help to enhance a tactile feedback provided to the user through the atraumatic tip 150, the shaft 118, and/or the handle 110 while navigating the balloon dilation device 100 through the anatomy of the patient to the Eustachian tube. The shoulder 560 can also limit proximal displacement of the atraumatic tip 150 relative to the distal end 122 of the shaft 118 to ensure that the tip 150 is always located beyond or at least partially beyond the distal end 122. The atraumatic tip 150 can be positioned beyond or at least partially beyond the distal end 122 of the shaft 118 even when the balloon advancer member 152 is in the proximal-most position in the slot 156. It is additionally or alternatively possible to ensure that tip 150 is located beyond or at least partially beyond the distal end 122 by controlling a dimensional stack-up of dimensions and anticipated dimensional variances relevant to the location of atraumatic tip 150 relative to the distal end 122. Example dimensions may include an axial length L2 of a first sleeve portion 670 of the sleeve 146 and an axial length L3 of a second sleeve portion 772 of the sleeve 146 and possible variances thereof. Other example dimensions that may be considered in the dimensional stack-up include an axial length of the shaft 118, a mounting location of the shaft 118 within the handle 110, a mounting location of the balloon advancer member 152 on the sleeve 146, and a length of the slot 156 and possible variances thereof. Yet another additional or alternative approach to ensure that the atraumatic tip 150 is always located beyond or at least partially beyond the distal end 122 can be to provide an engagement between a heated die used to form the atraumatic tip 150 and the distal end 122 of the shaft 118, with the engagement limiting travel of the die toward the distal end 122. The die can thus be used to define and control the location of the atraumatic tip 150 relative to the distal end 122. Yet another additional or alternative approach to ensure that the atraumatic tip 150 is always located beyond or at least partially beyond the distal end 122 can be to fix the strengthening sleeve of the shaft 118 to the core portion of the shaft 118 when a proximal surface of the atraumatic tip 15 is in engagement with the distal end 122 of the shaft 118.

Additionally or alternatively, the internal shoulder 560 of the inflatable balloon 126 can provide a stop against moving the inflatable balloon 126 past the proximal-most position. This stop function of the internal shoulder 560 can be provided in addition or alternative to a stop function provided by a proximal end of the slot 156, as described above.

As shown in FIG. 5B, when the inflatable balloon 126 is in a position that is distal of the proximal-most position, at least a portion of the inflatable balloon 126 can extend distally from the distal end 122 of the shaft 118. This can facilitate inserting the inflatable balloon 126 into the Eustachian tube with a lesser portion (or no portion) of the shaft 118 extending into the Eustachian tube. Due to the relative flexibility and softness of the inflatable balloon 126 when in the contracted state as compared to the relatively rigid hardness of the shaft 118, this can help position the balloon dilation device 100 in the Eustachian tube in a more atraumatic manner than implementations in which the inflatable balloon 126 does not extend distally beyond the distal end 122 of the shaft 118.

Although FIG. 5A shows the balloon advancer member 152 and the inflatable balloon 126 in the proximal-most positions and FIG. 5B shows the balloon advancer member 152 and the inflatable balloon 126 in the distal-most positions, the balloon advancer member 152 and the inflatable balloon 126 can be moved to one or more intermediate positions between the proximal-most positions and the distal-most positions in some instances.

As described above, the inflation lumen 142 defines a fluid channel between the inflation port 144 on the handle 110 and an interior of the inflatable balloon 126. As shown in FIGS. 1D and 5A-5B, the inflation lumen 142 can include a loop 162 that is configured to provide slack to the inflation lumen 142 such that the inflatable balloon 126 is movable relative to the shaft 118 while maintaining the inflatable balloon 126 in fluid communication with the inflation port 144. For instance, the loop 162 can have a first size (shown in FIG. 5A) when the inflatable balloon 126 is in the proximal-most position, the loop 162 can have a second size (shown in FIG. 5B) when the inflatable balloon 126 is in the distal-most position, and the first size can be greater than the second size.

The loop 162 can be elongated in a dimension that is substantially parallel to the longitudinal axis 236 (shown in FIG. 2) of the shaft 118. This can help to reduce (or prevent) kinking by making it less likely that a radius of the loop 162 will be reduced with displacement of the balloon in the distal direction. However, in other examples, the loop 162 can have a different shape (e.g., a circular shape) so long as an initial radius of the loop 162 is sufficient to accommodate an anticipated balloon displacement without a risk of the loop kinking.

Additionally, as shown in FIGS. 1D and 5A-5B, the handle 110 can include a retainer 164 that is configured to retain the loop 162 in an interior space defined by the retainer 164. The retainer 164 can help to maintain the loop 162 of the inflation lumen 142 in axial alignment as the loop 162 expands and contracts during movement of the inflatable balloon 128.

In FIG. 1D, the retainer 164 can include a plurality of side walls 164A-164B that extend around at least a portion of a periphery of the loop 162. For instance, the retainer 164 can include a proximal side wall 164A, a distal side wall 164B, and a lateral side wall 164C that extends between the proximal side wall 164A and the distal side wall 164B. In FIG. 1D, when the retainer 164 is coupled to the handle 110 in the internal cavity 116 (e.g., via a friction fit coupling, a welding coupling, and/or an adhesive coupling), the retainer 164 encloses the loop 162 on at least three sides. In FIG. 1D, the handle 110 can further enclose the loop 162 on the other three sides. In this arrangement, the loop 162 can be substantially enclosed within the interior space defined by the retainer 164 and the portion of the handle 110 to which the retainer 164 is coupled.

As shown in FIG. 1D, the side walls 164A-164C include a proximal aperture and a distal aperture that can provide for inflation lumen 142 extending through the interior space defined by the retainer 164. For instance, the proximal side wall 164A can include the proximal aperture and the distal side wall 164B can include the distal aperture. The inflation lumen 142 can extend from the inflation port 144 into the interior space of the retainer 164 through the proximal aperture, and the inflation lumen 142 can extend from the interior space of the retainer 164 to the distal end 114 of the handle 110 through the distal aperture.

Additionally, as shown in FIG. 1D, the balloon dilation device 149 can include a tube holder 166 that can assist in maintaining the inflation lumen 142 along an axis that is parallel to the longitudinal axis 236 of the shaft 118 at the tube holder 166. For instance, the shaft 118 can extend through a first aperture in the tube holder 166, the inflation lumen 142 can extend through a second aperture in the tube holder 166, and the first aperture and the second aperture can have respective axes that are parallel to each other. To accommodate movement of the inflatable balloon 126, the inflation lumen 142 can be slidable within the second aperture of the tube holder 166. The shaft 118 can be fixedly coupled to the tube holder 166 such that the shaft 118 cannot move relative to the tube holder 166. The tube holder 166 also can be fixedly coupled to the handle 110 (e.g., at a receptacle 168 in the internal cavity 116 of the handle 110. In this arrangement, the tube holder 166 can assist in fixedly coupling the shaft 118 to the handle 110 such that the shaft 118 is fixed to the tube holder 166 which is in turn fixed with respect to the handle 110.

Referring to FIG. 1D, a method of manufacturing the balloon dilation device 100 can include forming a sub-assembly including the shaft 118, the sleeve 146, the inflatable balloon 126, the support tube 154, the balloon advancer member 152, the inflation port 144, and/or the tube holder 166. The method can also include positioning the sub-assembly in a first segment of the handle 110. This can include positioning the tube holder 166 in the receptacle 168, positioning the balloon advancer member 152 in a portion the slot 156 of the first segment of the handle 110, and/or positioning the inflation port 144 at the proximal end 112 of the handle 110.

The method can further include coupling the retainer 164 to the first segment of the handle 110 with the loop 162 of the inflation lumen 142 in the interior space defined at least in part by the retainer 164. Coupling the retainer 164 to the handle 110 can also include positioning the inflation lumen 142 through the proximal aperture and the distal aperture of the retainer 164. The process can then include coupling the second segment of the handle 110 to the first segment of the handle 110 to substantially enclose the sub-assembly in the internal cavity 116 of the handle 110.

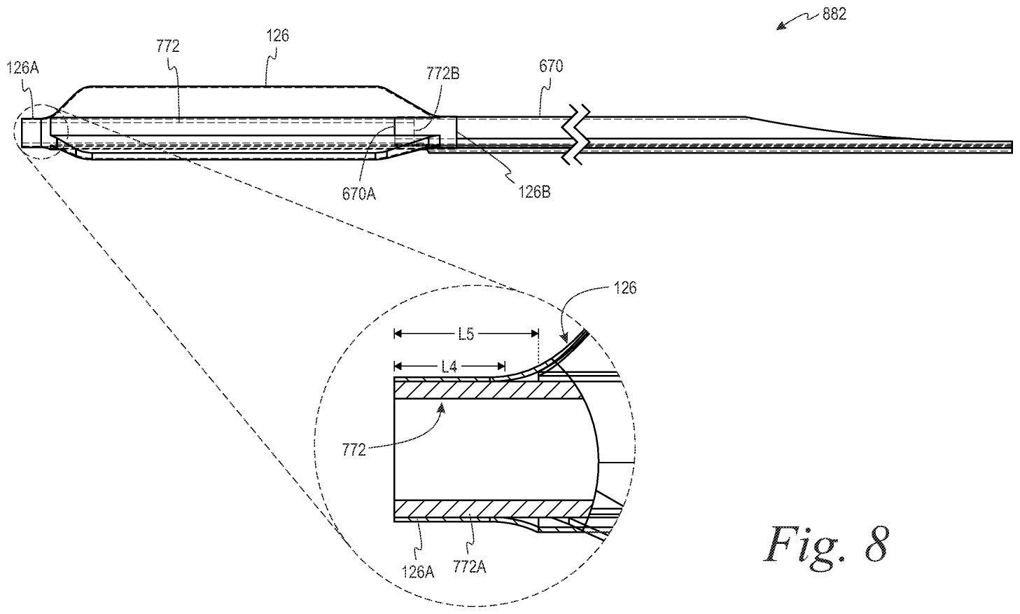

Additional aspects of a method of manufacturing the balloon dilation device 100 are shown in FIGS. 6A-9. In particular, FIGS. 6A-9 depict aspects of a process for forming and assembling the sleeve 146 and the inflatable balloon 126 to of the balloon dilation device 100, according to an example. In this example, the sleeve 146 includes the first sleeve portion 670 shown in FIGS. 6A-6D and the second sleeve portion 772 shown in FIGS. 7A-7B.

FIG. 6A depicts a side view of the first sleeve portion 670, FIG. 6B depicts an end view of a distal end 670A of the first sleeve portion 670, FIG. 6C depicts an end view of the a proximal end 670B of first sleeve portion 670, and FIG. 6D depicts an enlarged side view of the distal end 670A and the proximal end 670B of first sleeve portion 670. As shown in FIGS. 6B and 6C, the first sleeve portion 670 includes the inflation lumen 142 and the rider lumen 148. The inflation lumen 142 extends entirely through the first sleeve portion 670 to provide for flow of the fluid through the first sleeve portion 670 to the inflatable balloon 126. The rider lumen 148 also extends entirely through the first sleeve portion 670 to allow the shaft 118 to extend through the first sleeve portion 670 and/or define a portion of the airflow passageway. In one example, the first sleeve portion 670 can be formed by an extrusion process to form the first sleeve portion 670 as a dual lumen extrusion component, including the inflation lumen 142 and the rider lumen 148.

As shown in FIGS. 6A and 6D, the proximal end 670B of the first sleeve portion 670 includes a skived portion 674. The skived portion 674 can help to couple the proximal end 670B of the first sleeve portion 670 to the proximal section 146A of the sleeve 146 shown in FIG. 1D. In one implementation, the skived portion 674 can be formed by removing material of the first sleeve portion 670 adjacent the skived portion 674 using a cutting tool (e.g., a razor blade). For instance, the skived portion 674 can be formed by skiving away a part of the first sleeve portion 670 that defines the rider lumen 148 near the proximal end 670B. As such, a proximal opening of the rider lumen 148 can be offset distally from the proximal end 670B of the first sleeve portion 670. As one example, a length L1 of the skived portion 674 (e.g., defining an offset distance between the proximal end 670B and the proximal opening of the rider lumen 148 of the first sleeve portion 670) can be at least 0.5 inch.

In some implementations, after initially skiving the first sleeve portion 670, the skived portion 674 can have a crescent shape at the proximal end 670B, and the process can optionally include reshaping the skived portion 674 and/or the proximal end 670B to have a more circular shape. This can configure the proximal end 670B to have a shape that can better conform to a shape of a tube defining the proximal section 146A, and thereby facilitate coupling the first sleeve portion 670 to the proximal section 146A of the sleeve 146.

In one example, a length L2 of the first sleeve portion 670 between the distal end 670A and the proximal end 670B can be approximately 6.9 inch to approximately 7.3 inch (e.g., approximately 7.25 inch). Additionally, in one example, the skived portion 674 can be defined at least in part by a radius of curvature of approximately 3.25 inch relative to a dimension parallel to the length L2. Further, in an example, the skived portion 674 can be configured such that the proximal end has a thickness T1 (e.g, in a dimension perpendicular to the length) of approximately 0.30 inch.

As shown in FIG. 6D, the first sleeve portion 670 can include a second skived portion 676. The second skived portion 676 can be formed by removing a part of the first sleeve portion 670 that forms the inflation lumen 142 at the distal end 670A. As such, a distal opening of the inflation lumen 142 of the first sleeve portion 670 can be offset proximally from the distal end 670A of the first sleeve portion 670. The second skived portion 676 can help to couple the distal end 670A of the first sleeve portion 670 to the second sleeve portion 772 shown in FIGS. 7A-7B, and/or provide for the distal opening of the inflation lumen 142 communicating with an interior of the inflatable balloon 126, as described in further detail below.

As shown in FIGS. 6A and 6D, the second skived portion 676 can expose a lateral surface 678 of the rider lumen 148 at the distal end 670A. The lateral surface 678 can provide a surface area for coupling the distal end 670A of the first sleeve portion 670 to the second sleeve portion 772 (e.g., by heating bonding). As one example, a length L3 of the lateral surface 678 and the second skived portion 676 (e.g., defining an offset distance between the distal end 670A and the distal opening of the inflation lumen 142 of the first sleeve portion 670) can be approximately 0.08 inch.

Also, as shown in FIGS. 6A and 6D, the second skived portion 676 can include a proximal wall 680 that is oriented such that an angle 682 between the lateral surface 678 and the proximal wall 680 is greater than 90 degrees. For instance, in one example, the angle 682 between the lateral surface 678 and the proximal wall 680 can be approximately 120 degrees.

FIG. 7A depicts a side view of the second sleeve portion 772 and FIG. 7B depicts an end view of the second sleeve portion 772, according to an example. As shown in FIG. 7A-7B, the second sleeve portion 772 can include the rider lumen 148 that extends entirely between a distal end 772A of the second sleeve portion 772 and a proximal end 772B of the second sleeve portion 772. The rider lumen 148 can extend entirely through the second sleeve portion 772 to allow the shaft 118 to extend in the second sleeve portion 772 and/or define a portion of the airflow passageway. In an example, the second sleeve portion 772 can have a shape of a tube. Additionally, as an example, the second sleeve portion 772 can have a length L3 of approximately 1 inch. In one example, the second sleeve portion 772 can be formed by an extrusion process and cut to a predetermined length. Additionally, for example, the second sleeve portion 772 can be formed as a tri-layer extrusion.

FIG. 8 depicts an assembly 882 of the first sleeve portion 670, the second sleeve portion 772, and the inflatable balloon 126, according to an example. As shown in FIG. 8, the proximal end 772B of the second sleeve portion 772 can be coupled to the distal end 670A of the first sleeve portion 670. For example, the proximal end 772B of the second sleeve portion 772 can receive the distal end 670A of the first sleeve portion 670 such that respective portions of the first sleeve portion 670 and the second sleeve portion 772 overlap each other (e.g., at the lateral surface 678). As an example, after inserting the distal end 670A of the first sleeve portion 670 into the proximal end 772B of the second sleeve portion 772, the first sleeve portion 670 and the second sleeve portion 772 can be coupled to each other by a heat bonding.

In this arrangement, when the first sleeve portion 670 is coupled to the second sleeve portion 772, the distal opening of the inflation lumen 142 of the first sleeve portion 670 remains unobstructed. As shown in FIG. 8, the inflatable balloon 126 can be coupled to the sub-assembly of the first sleeve portion 670 and the second sleeve portion 772. For instance, a distal end 126A of the inflatable balloon 126 can be coupled to the distal end 772A of the second sleeve portion 772, and a proximal end 126B of the inflatable balloon 126 can be coupled to the first sleeve portion 670.

As shown in FIG. 8, the proximal end 126B of the inflatable balloon 126 can be coupled to the first sleeve portion 670 at a position that is proximal of the second skived portion 676. In this arrangement, the distal end of the inflation lumen 142 is located in the interior of the inflatable balloon 126. In one example, the inflatable balloon 126 can extend around the first sleeve portion 670 and can be heat bonded to the first sleeve portion 670 around an entire circumference of the first sleeve portion 670. This can form a proximal seal to help retain fluid in the interior of the inflatable balloon 126 when the inflatable balloon 126 is inflated.

The distal end 126A of the inflatable balloon 126 can be coupled to the distal end 772A of the second sleeve portion 772. For example, as shown in FIG. 8, the distal end 126A of the inflatable balloon 126 extends around an entire circumference of the second sleeve portion 772, and is heat bonded to the second sleeve portion 772. This can form a distal seal to help retain fluid in the interior of the inflatable balloon 126 when the inflatable balloon 126 is inflated. In one example, the distal end 126A of the inflatable balloon 126 can be bonded to the distal end 772A of the second sleeve portion 772 over a length L4 of approximately 0.06 mm.

In some examples, a process for forming the subassembly 882 shown in FIG. 8 can include coupling the first sleeve portion 670 to the second sleeve portion 772, sliding the inflatable balloon 126 over the first sleeve portion 670 and the second sleeve portion 772 such that the distal end of the inflation lumen 142 is located in the interior of the inflatable balloon 126, coupling the proximal end 126B of the inflatable balloon 126 to the first sleeve portion 670, and coupling the distal end 126A of the inflatable balloon 126 to the second sleeve portion 772.

The process can additionally include cutting the inflatable balloon 126 and the second sleeve portion 772 at the distal end to remove excess material and/or align the distal end 126A of the inflatable balloon 126 and the distal end 772A of the second sleeve portion 772. For instance, in one example, the inflatable balloon 126 and the second sleeve portion 772 can be cut such that the distal end 126A of the inflatable balloon 126 has a length L5 of approximately 0.7 mm.

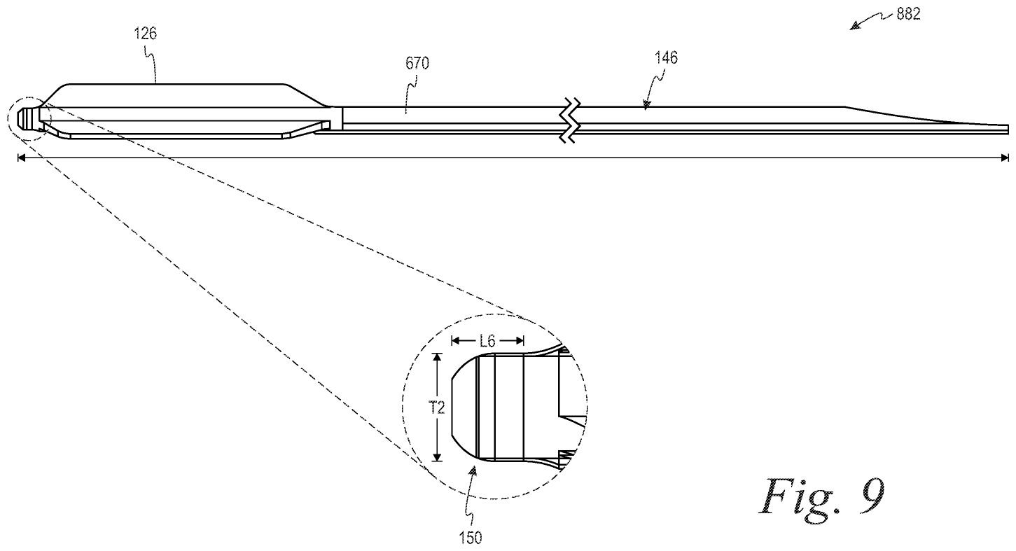

Cutting the inflatable balloon 126 and the second sleeve portion 772 to a predetermined length can additionally or alternatively help to facilitate forming the atraumatic tip 150 at the distal end 126A of the inflatable balloon 126. FIG. 9 shows the subassembly 882 after the distal end 126A of the inflatable balloon 126 and the distal end 772A of the second sleeve portion 772 have been formed into the atraumatic tip 150. In one example, the distal end 126A of the inflatable balloon 126 and the distal end 772A of the second sleeve portion 772 can be pressed into a heated die to form a rounded shape (e.g., defined by a radius). In one implementation, after forming the atraumatic tip 150 shown in FIG. 9, the distal end 126A of the inflatable balloon 126 can have a length L6 of approximately 0.06 mm and a thickness T2 of approximately 0.9 mm.

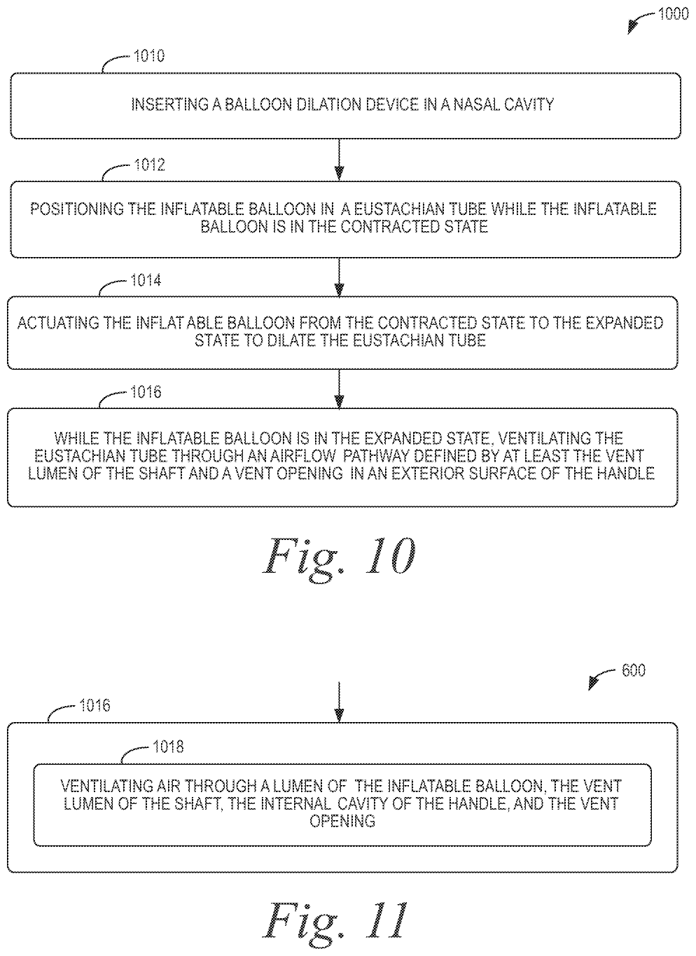

Referring now to FIG. 10, a flowchart for a process 1000 of using a balloon dilation device is shown according to an example. At block 1010, the process 1000 includes inserting a balloon dilation device in a nasal cavity. The balloon dilation device includes a handle having a proximal end and a distal end. The handle defines an internal cavity. The balloon dilation device also includes a shaft extending distally from the distal end of the handle. The shaft includes a vent lumen extending between a first opening at a distal end of the shaft and a second opening of the shaft. The second opening of the shaft is disposed in the internal cavity of the handle. Additionally, the balloon dilation device includes an inflatable balloon disposed about the distal end of the shaft. The inflatable balloon is configured to be actuated between a contracted state and an expanded state.

After inserting the balloon dilation device into the nasal cavity at block 1010, the process 1000 includes positioning the inflatable balloon in a Eustachian tube while the inflatable balloon is in the contracted state at block 1012. After positioning the inflatable balloon in the Eustachian tube at block 1012, the process 1000 includes actuating the inflatable balloon from the contracted state to the expanded state to dilate the Eustachian tube at block 1014. The process 1000 also includes, while the inflatable balloon is in the expanded state, ventilating the Eustachian tube through an airflow pathway defined by at least the vent lumen of the shaft and a vent opening in an exterior surface of the handle at block 1016. The vent lumen and the vent opening are configured such that the airflow pathway defines a tortuous path that inhibits access to the vent lumen.

FIGS. 11-16 depict additional aspects of the process 1000 according to further examples. As shown in FIG. 11, ventilating the Eustachian tube through the airflow pathway at block 1016 can include ventilating air through a lumen of the inflatable balloon, the vent lumen of the shaft, the internal cavity of the handle, and the vent opening at block 1018.

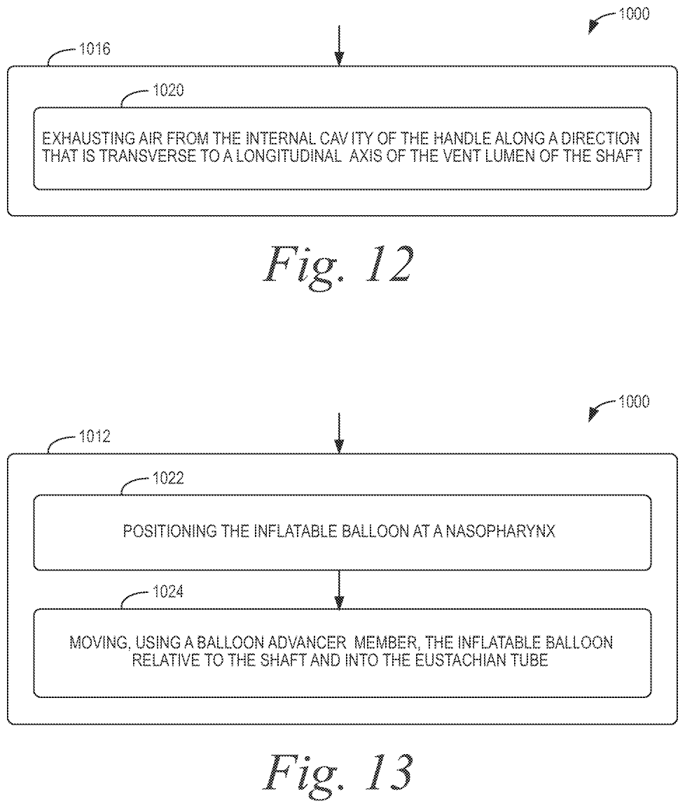

As shown in FIG. 12, ventilating the Eustachian tube through the airflow pathway at block 1016 can include exhausting air from the internal cavity of the handle along a direction that is transverse to a longitudinal axis of the vent lumen of the shaft at block 1020.

As shown in FIG. 13, positioning the inflatable balloon in the Eustachian tube at block 1012 can include (i) positioning the inflatable balloon at a Eustachian tube opening in a nasopharynx at block 1022, and moving, using a balloon advancer member, the inflatable balloon relative to the shaft and into the Eustachian tube at block 1024.

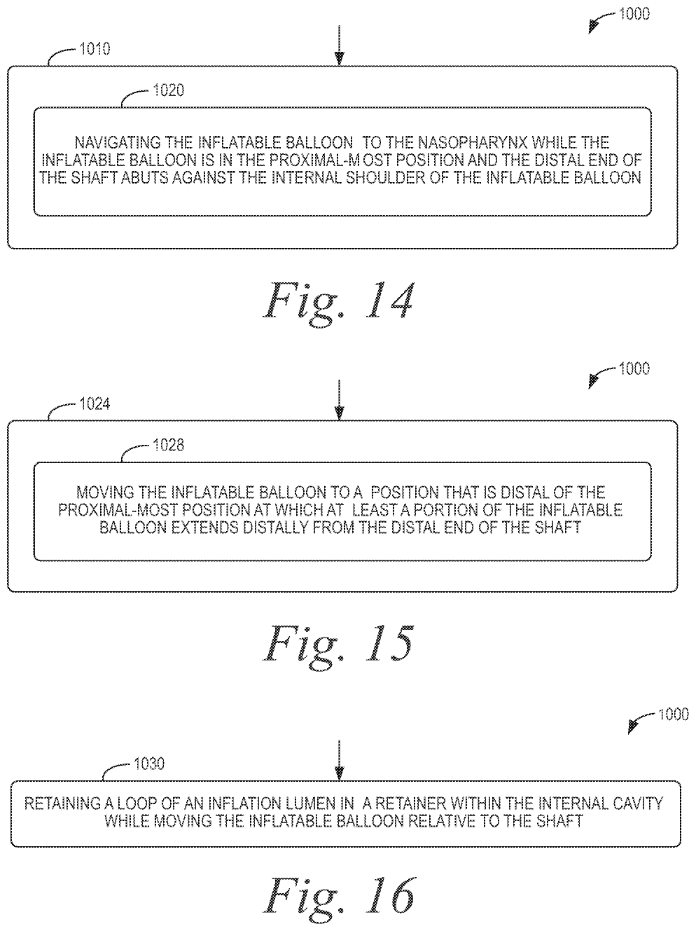

In example shown in FIG. 14, the inflatable balloon is moveable between a distal-most position and a proximal-most position relative to the shaft, and the inflatable balloon includes an internal shoulder that abuts against the distal end of the shaft when the inflatable balloon is in the proximal-most position. As shown in FIG. 14, inserting the balloon dilation device in the nasal cavity at block 1010 can include navigating the inflatable balloon to the Eustachian tube opening in the nasopharynx while the inflatable balloon is in the proximal-most position and the distal end of the shaft abuts against the internal shoulder of the inflatable balloon at block 1026.

As shown in FIG. 15, moving the inflatable balloon at block 1024 can include moving the inflatable balloon to a position that is distal of the proximal-most position at which at least a portion of the inflatable balloon extends distally from the distal end of the shaft at block 1028.

As shown in FIG. 16, the process 1000 can also include retaining a loop of an inflation lumen in a retainer within the internal cavity while moving the inflatable balloon relative to the shaft at block 1030. In this example, the inflation lumen extends from an inflation port of the handle to the inflatable balloon.

Further, the disclosure comprises examples according to the following clauses:

Clause 1: A balloon dilation device includes a handle having a proximal end and a distal end. The handle defines an internal cavity. The balloon dilation device also includes a shaft extending distally from the distal end of the handle. The shaft includes a vent lumen extending between a first opening at a distal end of the shaft and a second opening of the shaft. The second opening of the shaft is disposed in the internal cavity of the handle. Additionally, the balloon dilation device includes an inflatable balloon disposed about the distal end of the shaft. The inflatable balloon is configured to be actuated between a contracted state and an expanded state. The balloon dilation device also includes an inflation lumen extending from an inflation port of the handle to the inflatable balloon. At least the vent lumen of the shaft and a vent opening in an exterior surface of the handle define an airflow pathway between the first opening and the vent opening.

Clause 2: The balloon dilation device of Clause 1, wherein the vent lumen and the vent opening are configured such that the airflow pathway defines a tortuous path that inhibits access to the vent lumen.

Clause 3: A balloon dilation device includes a handle having a proximal end and a distal end. The handle defines an internal cavity. The balloon dilation device also includes a shaft extending distally from the distal end of the handle. The shaft includes a vent lumen extending between a first opening at a distal end of the shaft and a second opening of the shaft. The second opening of the shaft is disposed in the internal cavity of the handle. Additionally, the balloon dilation device includes an inflatable balloon disposed about the distal end of the shaft. The inflatable balloon is configured to be actuated between a contracted state and an expanded state. The balloon dilation device also includes an inflation lumen extending from an inflation port of the handle to the inflatable balloon. At least the vent lumen of the shaft and a vent opening in an exterior surface of the handle define an airflow pathway for ventilating an Eustachian tube while the inflatable balloon is in the expanded state. The vent lumen and the vent opening are configured such that the airflow pathway defines a tortuous path that inhibits access to the vent lumen.

Clause 4: The balloon dilation device of any one of Clauses 1-3, wherein the second opening of the shaft is at the proximal end of the shaft.

Clause 5: The balloon dilation device of any one of Clauses 1-4, wherein a longitudinal axis of the vent lumen is transverse to the airflow pathway at the vent opening in the exterior surface of the handle.

Clause 6: The balloon dilation device of any one of Clauses 1-5, wherein the second opening of the vent lumen is proximal of the vent opening in the exterior surface of the handle.

Clause 7: The balloon dilation device of any one of Clauses 1-6, wherein the vent opening is disposed between the proximal end of the handle and the distal end of the handle.

Clause 8: The balloon dilation device of Clause 7, wherein the vent opening is nearer to the distal end of the handle than the proximal end of the handle.

Clause 9: The balloon dilation device of any one of Clauses 1-8, wherein the airflow pathway is further defined by the internal cavity of the handle.

Clause 10: The balloon dilation device of any one of Clauses 1-9, further including balloon advancer member that is configured to move the inflatable balloon relative to the shaft.

Clause 11: The balloon dilation device of Clause 10, wherein the balloon advancer member is configured to move within a slot in the exterior surface of the handle, and wherein the slot in the exterior surface of the handle defines the vent opening in the exterior surface of the handle.

Clause 12: The balloon dilation device of any one of Clauses 10-11, wherein the balloon advancer member is coupled to a sleeve disposed around the shaft, and the sleeve is coupled to the inflatable balloon.

Clause 13: The balloon dilation device of any one of Clauses 10-12, wherein the inflatable balloon is moveable between a distal-most position and a proximal-most position relative to the shaft, and wherein the inflatable balloon includes an internal shoulder that abuts against the distal end of the shaft when the inflatable balloon is in the proximal-most position.

Clause 14: The balloon dilation device of Clause 13, wherein, when the inflatable balloon is in a position that is distal of the proximal-most position, at least a portion of the inflatable balloon extends distally from the distal end of the shaft.

Clause 15: The balloon dilation device of any one of Clauses 1-14, wherein the inflation lumen is external to the vent lumen of the shaft.

Clause 16: The balloon dilation device of any one of Clauses 1-15, wherein the inflation lumen defines a fluid channel between the inflation port on the handle and an interior of the inflatable balloon, wherein the inflation lumen includes a loop that is configured to provide slack to the inflation lumen such that the inflatable balloon is movable relative to the shaft while maintaining the inflatable balloon in fluid communication with the inflation port.

Clause 17: The balloon dilation device of Clause 16, wherein the handle includes a retainer that is configured to retain the loop in an interior space defined by the retainer.

Clause 18: The balloon dilation device of Clause 17, wherein the retainer includes a plurality of side walls that extend around at least a portion of a periphery of the loop.

Clause 19: The balloon dilation device of Clause 18, wherein the plurality of side walls includes a proximal aperture and a distal aperture, wherein the inflation lumen extends from the inflation port into the interior space of the retainer through the proximal aperture, and wherein the inflation lumen extends from the interior space of the retainer to the distal end of the handle through the distal aperture.

Clause 20: The balloon dilation device of any one of Clauses 16-19, wherein the loop is elongated in a dimension that is substantially parallel to a longitudinal axis of the shaft.

Clause 21: The balloon dilation device of any one of Clauses 1-20, further including an atraumatic tip at a distalmost end of the inflatable balloon, and wherein the atraumatic tip and the inflatable balloon are integrally formed as a single, monolithic structure.

Clause 22: The balloon dilation device of Clause 21, wherein the atraumatic tip further defines the airflow pathway.

Clause 23: The balloon dilation device of Clause 22, wherein the inflatable balloon further defines the airflow pathway.

Clause 24: The balloon dilation device of any one of Clauses 1-23, wherein a distal portion of the shaft includes a curved portion.

Clause 25: The balloon dilation device of Clause 24, wherein the shaft is non-malleable at the curved portion.

Clause 26: The balloon dilation device of any one of Clauses 24-25, wherein the distal portion of the shaft further includes a straight segment that is distal of the curved portion.

Clause 27: The balloon dilation device of Clause 26, wherein the straight segment has a length of approximately 9 millimeters.

Clause 28: The balloon dilation device of any one of Clauses 24-27, wherein the curved portion has a radius of curvature of approximately one inch.

Clause 29: The balloon dilation device of any one of Clauses 24-28, wherein the curved portion is configured such that the distal end of the shaft is offset from a longitudinal axis of the shaft by approximately 6 millimeters to approximately 10 millimeters.

Clause 30: The balloon dilation device of any one of Clauses 1-29, wherein the inflatable balloon has a cylindrical shape when the inflatable balloon is inflated.

Clause 31: The balloon dilation device of any one of Clauses 1-30, wherein the inflation lumen is defined by a sleeve having a first end that is fluidically coupled to the inflation port and a second end that is fluidically coupled to the inflatable balloon.

Clause 32: The balloon dilation device of Clause 31, wherein the sleeve comprises a first sleeve portion coupled to a second sleeve portion, wherein the first sleeve portion comprises the inflation lumen and a rider lumen, wherein the shaft is in the rider lumen, wherein a proximal end of the second sleeve portion is coupled to a distal end of the first sleeve portion, wherein a proximal end of the inflatable balloon is coupled to the first sleeve portion such that a distal end of the inflation lumen is in an interior of the inflatable balloon, and wherein a distal end of the inflatable balloon is coupled to the second sleeve portion.

Clause 33: The balloon dilation device of Clause 32, wherein the first sleeve portion comprises a skived portion that is configured to couple to a tube of a proximal portion of the sleeve.

Clause 34: The balloon dilation device of any one of Clauses 32-33, wherein the distal end of the inflatable balloon and the distal end of the second sleeve portion define an atraumatic tip have a rounded shape.

Clause 35: A method of using a balloon dilation device includes inserting a balloon dilation device in a nasal cavity. The balloon dilation device includes a handle having a proximal end and a distal end. The handle defines an internal cavity. The balloon dilation device also includes a shaft extending distally from the distal end of the handle. The shaft includes a vent lumen extending between a first opening at a distal end of the shaft and a second opening of the shaft. The second opening of the shaft is disposed in the internal cavity of the handle. Additionally, the balloon dilation device includes an inflatable balloon disposed about the distal end of the shaft. The inflatable balloon is configured to be actuated between a contracted state and an expanded state. The method also includes, after inserting the balloon dilation device into the nasal cavity, positioning the inflatable balloon in a Eustachian tube while the inflatable balloon is in the contracted state. The method further includes, after positioning the inflatable balloon in the Eustachian tube, actuating the inflatable balloon from the contracted state to the expanded state to dilate the Eustachian tube. The method also includes, while the inflatable balloon is in the expanded state, ventilating the Eustachian tube through an airflow pathway defined by at least the vent lumen of the shaft and a vent opening in an exterior surface of the handle. The vent lumen and the vent opening are configured such that the airflow pathway defines a tortuous path that inhibits access to the vent lumen.

Clause 36: The method of Clause 35, wherein ventilating the Eustachian tube through the airflow pathway includes ventilating air through a lumen of the inflatable balloon, the vent lumen of the shaft, the internal cavity of the handle, and the vent opening.

Clause 37: The method of any one of Clauses 35-36, wherein ventilating the Eustachian tube through the airflow pathway includes exhausting air from the internal cavity of the handle along a direction that is transverse to a longitudinal axis of the vent lumen of the shaft.

Clause 38: The method of any one of Clauses 35-37, wherein positioning the inflatable balloon in the Eustachian tube includes positioning the inflatable balloon at a Eustachian tube opening in a nasopharynx, and moving, using a balloon advancer member, the inflatable balloon relative to the shaft and into the Eustachian tube.

Clause 39: The method of Clause 38, wherein the inflatable balloon is moveable between a distal-most position and a proximal-most position relative to the shaft, wherein the inflatable balloon includes an internal shoulder that abuts against the distal end of the shaft when the inflatable balloon is in the proximal-most position, and wherein inserting the balloon dilation device in the nasal cavity includes navigating the inflatable balloon to the Eustachian tube opening in the nasopharynx while the inflatable balloon is in the proximal-most position and the distal end of the shaft abuts against the internal shoulder of the inflatable balloon.

Clause 40: The method of Clause 39, wherein moving the inflatable balloon includes moving the inflatable balloon to a position that is distal of the proximal-most position at which at least a portion of the inflatable balloon extends distally from the distal end of the shaft.

Clause 41: The method of any one of Clauses 38-40, further including retaining a loop of an inflation lumen in a retainer within the internal cavity while moving the inflatable balloon relative to the shaft, wherein the inflation lumen extends from an inflation port of the handle to the inflatable balloon.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present application is not to be limited by the subject specification, but rather only by the plain meaning of the claim terms employed.

What is claimed is:

1. A balloon dilation device comprising:
a handle having a proximal end and a distal end, wherein the handle defines an internal cavity;
a shaft extending distally from the distal end of the handle, wherein the shaft comprises a vent lumen extending between a first opening at a distal end of the shaft and a second opening of the shaft, wherein the second opening of the shaft is disposed in the internal cavity of the handle;
an inflatable balloon disposed about the distal end of the shaft, wherein the inflatable balloon is configured to be actuated between a contracted state and an expanded state; and
an inflation lumen extending from an inflation port of the handle to the inflatable balloon,
wherein at least the vent lumen of the shaft and a vent opening in an exterior surface of the handle define an airflow pathway between the first opening and the vent opening.

2. The balloon dilation device of claim 1, wherein the vent lumen and the vent opening are configured such that the airflow pathway defines a tortuous path that inhibits access to the vent lumen.

3. A balloon dilation device comprising:
a handle having a proximal end and a distal end, wherein the handle defines an internal cavity;
a shaft extending distally from the distal end of the handle, wherein the shaft comprises a vent lumen extending between a first opening at a distal end of the shaft and a second opening of the shaft, wherein the second opening of the shaft is disposed in the internal cavity of the handle;
an inflatable balloon disposed about the distal end of the shaft, wherein the inflatable balloon is configured to be actuated between a contracted state and an expanded state; and
an inflation lumen extending from an inflation port of the handle to the inflatable balloon,
wherein at least the vent lumen of the shaft and a vent opening in an exterior surface of the handle define an airflow pathway for ventilating an Eustachian tube while the inflatable balloon is in the expanded state, and
wherein the vent lumen and the vent opening are configured such that the airflow pathway defines a tortuous path that inhibits access to the vent lumen.

4. The balloon dilation device of any one of claims 1-3, wherein the second opening of the shaft is at the proximal end of the shaft.

5. The balloon dilation device of any one of claims 1-4, wherein a longitudinal axis of the vent lumen is transverse to the airflow pathway at the vent opening in the exterior surface of the handle.

6. The balloon dilation device of any one of claims 1-5, wherein the second opening of the vent lumen is proximal of the vent opening in the exterior surface of the handle.

7. The balloon dilation device of any one of claims 1-6, wherein the vent opening is disposed between the proximal end of the handle and the distal end of the handle.

8. The balloon dilation device of claim 7, wherein the vent opening is nearer to the distal end of the handle than the proximal end of the handle.

9. The balloon dilation device of any one of claims 1-8, wherein the airflow pathway is further defined by the internal cavity of the handle.

10. The balloon dilation device of any one of claims 1-9, further comprising balloon advancer member that is configured to move the inflatable balloon relative to the shaft.

11. The balloon dilation device of claim 10, wherein the balloon advancer member is configured to move within a slot in the exterior surface of the handle, and
wherein the slot in the exterior surface of the handle defines the vent opening in the exterior surface of the handle.

12. The balloon dilation device of any one of claims 10-11, wherein the balloon advancer member is coupled to a sleeve disposed around the shaft, and the sleeve is coupled to the inflatable balloon.

13. The balloon dilation device of any one of claims 10-12, wherein the inflatable balloon is moveable between a distal-most position and a proximal-most position relative to the shaft, and
wherein the inflatable balloon comprises an internal shoulder that abuts against the distal end of the shaft when the inflatable balloon is in the proximal-most position.

14. The balloon dilation device of claim 13, wherein, when the inflatable balloon is in a position that is distal of the proximal-most position, at least a portion of the inflatable balloon extends distally from the distal end of the shaft.

15. The balloon dilation device of any one of claims 1-14, wherein the inflation lumen is external to the vent lumen of the shaft.

16. The balloon dilation device of any one of claims 1-15, wherein the inflation lumen defines a fluid channel between the inflation port on the handle and an interior of the inflatable balloon, wherein the inflation lumen comprises a loop that is configured to provide slack to the inflation lumen such that the inflatable balloon is movable relative to the shaft while maintaining the inflatable balloon in fluid communication with the inflation port.

17. The balloon dilation device of claim 16, wherein the handle comprises a retainer that is configured to retain the loop in an interior space defined by the retainer.

18. The balloon dilation device of claim 17, wherein the retainer comprises a plurality of side walls that extend around at least a portion of a periphery of the loop.

19. The balloon dilation device of claim 18, wherein the plurality of side walls comprises a proximal aperture and a distal aperture,
wherein the inflation lumen extends from the inflation port into the interior space of the retainer through the proximal aperture, and
wherein the inflation lumen extends from the interior space of the retainer to the distal end of the handle through the distal aperture.

20. The balloon dilation device of any one of claims 16-19, wherein the loop is elongated in a dimension that is substantially parallel to a longitudinal axis of the shaft.

21. The balloon dilation device of any one of claims 1-20, further comprising an atraumatic tip at a distalmost end of the inflatable balloon,
wherein the atraumatic tip and the inflatable balloon are integrally formed as a single, monolithic structure.

22. The balloon dilation device of claim 21, wherein the atraumatic tip further defines the airflow pathway.

23. The balloon dilation device of claim 22, wherein the inflatable balloon further defines the airflow pathway.

24. The balloon dilation device of any one of claims 1-23, wherein a distal portion of the shaft comprises a curved portion.

25. The balloon dilation device of claim 24, wherein the shaft is non-malleable at the curved portion.

26. The balloon dilation device of any one of claims 24-25, wherein the distal portion of the shaft further comprises a straight segment that is distal of the curved portion.

27. The balloon dilation device of claim 26, wherein the straight segment has a length of approximately 9 millimeters.

28. The balloon dilation device of any one of claims 24-27, wherein the curved portion has a radius of curvature of approximately one inch.

29. The balloon dilation device of any one of claims 24-28, wherein the curved portion is configured such that the distal end of the shaft is offset from a longitudinal axis of the shaft by approximately 6 millimeters to approximately 10 millimeters.

30. The balloon dilation device of any one of claim 1-29, wherein the inflatable balloon has a cylindrical shape when the inflatable balloon is inflated.

31. The balloon dilation device of any one of claims 1-30, wherein the inflation lumen is defined by a sleeve having a first end that is fluidically coupled to the inflation port and a second end that is fluidically coupled to the inflatable balloon.

32. The balloon dilation device of claim 31, wherein the sleeve comprises a first sleeve portion coupled to a second sleeve portion,
wherein the first sleeve portion comprises the inflation lumen and a rider lumen, and
wherein the shaft is in the rider lumen,
wherein a proximal end of the second sleeve portion is coupled to a distal end of the first sleeve portion,
wherein a proximal end of the inflatable balloon is coupled to the first sleeve portion such that a distal end of the inflation lumen is in an interior of the inflatable balloon, and
wherein a distal end of the inflatable balloon is coupled to the second sleeve portion.

33. The balloon dilation device of claim 32, wherein the first sleeve portion comprises a skived portion that is configured to couple to a tube of a proximal portion of the sleeve.

34. The balloon dilation device of any one of claims 32-33, wherein the distal end of the inflatable balloon and the distal end of the second sleeve portion define an atraumatic tip have a rounded shape.

35. A method of using a balloon dilation device, comprising:
inserting a balloon dilation device in a nasal cavity, wherein the balloon dilation device comprises:
a handle having a proximal end and a distal end, wherein the handle defines an internal cavity,
a shaft extending distally from the distal end of the handle, wherein the shaft comprises a vent lumen extending between a first opening at a distal end of the shaft and a second opening of the shaft, wherein the second opening of the shaft is disposed in the internal cavity of the handle, and
an inflatable balloon disposed about the distal end of the shaft, wherein the inflatable balloon is configured to be actuated between a contracted state and an expanded state;
after inserting the balloon dilation device into the nasal cavity, positioning the inflatable balloon in a Eustachian tube while the inflatable balloon is in the contracted state;
after positioning the inflatable balloon in the Eustachian tube, actuating the inflatable balloon from the contracted state to the expanded state to dilate the Eustachian tube; and
while the inflatable balloon is in the expanded state, ventilating the Eustachian tube through an airflow pathway defined by at least the vent lumen of the shaft and a vent opening in an exterior surface of the handle,
wherein the vent lumen and the vent opening are configured such that the airflow pathway defines a tortuous path that inhibits access to the vent lumen.

36. The method of claim 35, wherein ventilating the Eustachian tube through the airflow pathway comprises ventilating air through a lumen of the inflatable balloon, the vent lumen of the shaft, the internal cavity of the handle, and the vent opening.

37. The method of any one of claims 35-36, wherein ventilating the Eustachian tube through the airflow pathway comprises exhausting air from the internal cavity of the handle along a direction that is transverse to a longitudinal axis of the vent lumen of the shaft.

38. The method of any one of claims 35-37, wherein positioning the inflatable balloon in the Eustachian tube comprises:
positioning the inflatable balloon at a Eustachian tube opening in a nasopharynx; and
moving, using a balloon advancer member, the inflatable balloon relative to the shaft and into the Eustachian tube.

39. The method of claim 38, wherein the inflatable balloon is moveable between a distal-most position and a proximal-most position relative to the shaft,
wherein the inflatable balloon comprises an internal shoulder that abuts against the distal end of the shaft when the inflatable balloon is in the proximal-most position, and
wherein inserting the balloon dilation device in the nasal cavity comprises navigating the inflatable balloon to the Eustachian tube opening in the nasopharynx while the inflatable balloon is in the proximal-most position and the distal end of the shaft abuts against the internal shoulder of the inflatable balloon.

40. The method of claim 39, wherein moving the inflatable balloon comprises moving the inflatable balloon to a position that is distal of the proximal-most position at which at least a portion of the inflatable balloon extends distally from the distal end of the shaft.

41. The method of any one of claims 38-40, further comprising retaining a loop of an inflation lumen in a retainer within the internal cavity while moving the inflatable balloon relative to the shaft, wherein the inflation lumen extends from an inflation port of the handle to the inflatable balloon.
